(19) United States
(12) Plant Patent Application Publication
(10) Pub. No.: US 2002/0129428 P1
(43) Pub. Date: Sep. 12, 2002

(54) GERANIUM PLANT NAMED 'FISBLIZDARK'

(75) Inventor: Angelika Utecht, Montabaur (DE)

Correspondence Address: Foley & Lardner, Suite 500, 3000 K Street, N.W., P.O. Box 25696, Washington, DC, 20007-8696, US

(73) Assignee: FLORFIS AG

(21) Appl. No.: 09/800,496

(22) Filed: Mar. 8, 2001

(51) Int. Cl. A01H 5/00

(52) U.S. Cl. PLT/332

(57) ABSTRACT

A new and distinct cultivar of geranium plant named ‘Fisblizdark’, as described and illustrated, and particularly characterized by the combined features of dark-red, single flowers, large umbels and rich flowering, intense green, slightly zoned foliage, and tall, trailing, though well-branched plant habit.

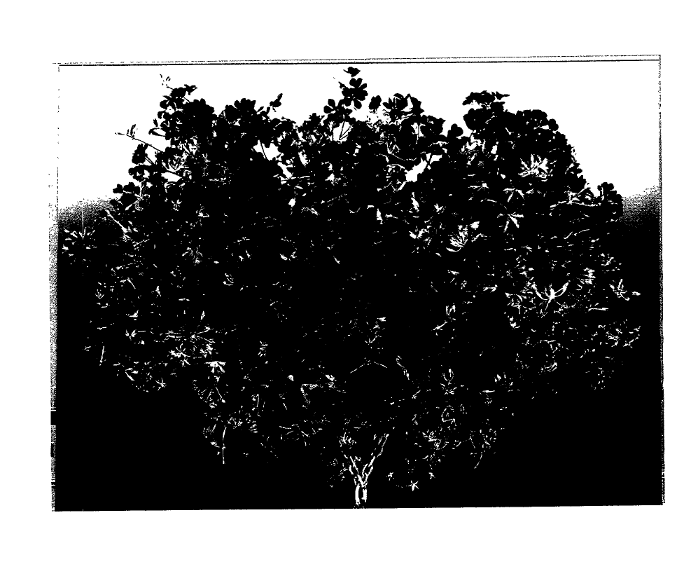

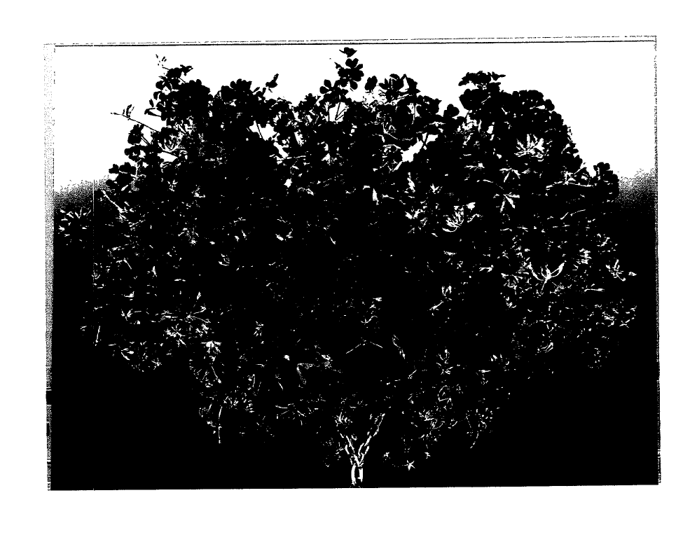

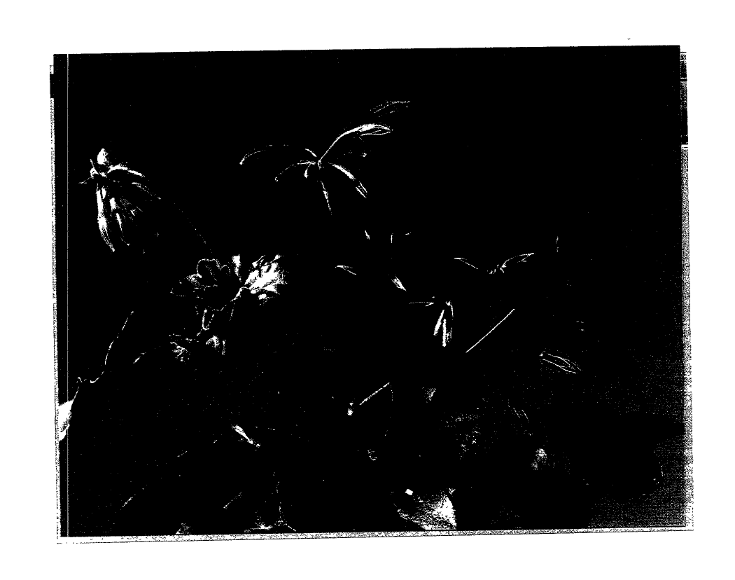

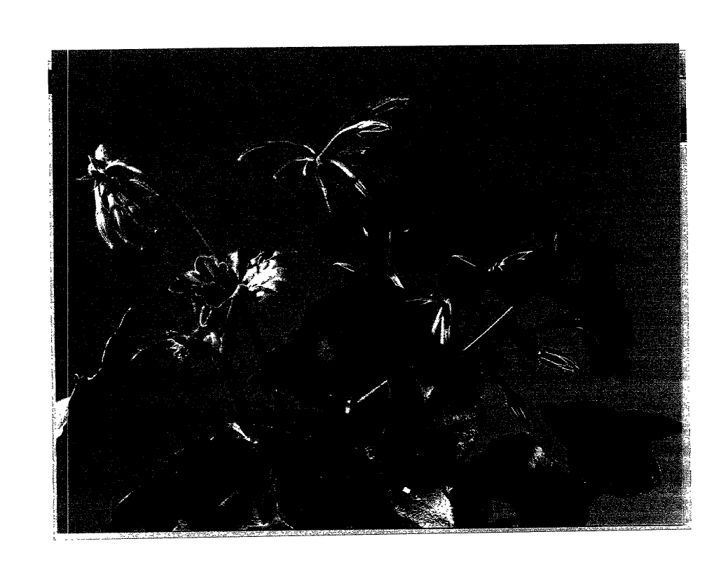

### BACKGROUND OF THE INVENTION

The present invention comprises a new and distinct cultivar of geranium, botanically known as Pelargonium peltatum, and hereinafter referred to by the cultivar name ‘Fisblizdark’. ‘Fisblizdark’ is a product of a planned breeding program which had the objective of creating new ivy geranium cultivars with single-type flowers, relatively vigorous, but well-branched growth habit, and in various flower colors. ‘Fisblizdark’ was originated from a hybridization made by the inventor Angelika Utecbt in a controlled breeding program in Galdar, Gran Canaria, Spain, in 1996.

The female parent was the variety ‘Fiscoral’, U.S. plant patent application Ser. No. 09/323,151, having orange-red, double flowers, fresh-green, slightly zoned foliage, and tall, well-branched plant habit. The male parent of ‘Fisblizdark’ was a hybrid seedling, no. 823/39, derived from a cross with ‘Fisrock’, U.S. Plant Pat. No. 10,365, and characterized by deep red single-type flowers, medium-green, zoned foliage, and medium to tall plant habit.

‘Fisblizdark’ was selected as one flowering plant within the progeny of the stated cross by Angelika Utecht in a controlled environment in Galdar, Gran Canaria, Spain, in 1997.

The first act of asexual reproduction of ‘Fisblizdark’ was accomplished when vegetative cuttings were taken from the initial selection in the fall of 1997 in a controlled environment in Galdar, Gran Canaria, Spain, by, or under the supervision of, Angelika Utecht. Horticultural examination of plants grown from these cuttings initiated in the spring of 1998 in Hillscheid, Germany, and continuing thereafter, has demonstrated that the combination of characteristics as herein disclosed for ‘Fisblizdark’ are firmly fixed and are retained through successive generations of asexual reproduction.

‘Fisblizdark’ has not been observed under all possible environmental conditions. The phenotype may vary significantly with variations in environment such as temperature, light intensity and day length.

### BRIEF DESCRIPTION OF THE INVENTION

The following observations, measurements, and comparisons describe plants grown in Hillscheid, Germany, and in Langley, British Columbia, Canada, under greenhouse conditions which approximate those generally used in commercial practice.

The following traits have been repeatedly observed and are determined to be basic characteristics of ‘Fisblizdark’, which in combination distinguish this geranium as a new and distinct cultivar

1. dark-red colored, round, single-type flowers;

2. large, flat semi-spherically shaped umbels;

3. relatively dark-green, smallish, and slightly zoned leaves;

4. vigorous growth and relatively tall, trailing, though well-branched plant habit; and

5. early spring flowering response.

Of the many commercial cultivars known to the present inventor, the most similar in comparison to ‘Fisblizdark’ is ‘Fizzard’, U.S. Plant Patent No. 10,992. In comparison to ‘Fizzard’, ‘Fisblizdark’ has a distinctly darker red flower color, slightly shorter upper petals and therefore more round shaped florets, and darker green, smaller, somewhat stronger zoned leaves with entire margins, while ‘Fizzard’ develops weak crenation near the tips. Furthermore, flowering response of ‘Fisblizdark’ is early, but not extremely early, as with ‘Fizzard’

### BRIEF DESCRIPTION OF THE DRAWING

The accompanying photographic drawings show typical flower and foliage characteristics of ‘Fisblizdark’ with colors being as true as possible will illustrations of this type. The measurements were taken in Langley, British Columbia, Canada, on Jul. 20, 2000. The plants were growing in 6 inch pots in a greenhouse, and had 15 weeks of cultivation time from the planting of rooted cuttings on Apr. 3, 2000. The plants had not been pinched.

In the following description color references are made to The Royal Horticultural Society Colour Chart (RHS). The color values were determined indoors from flowers developed in a greenhouse in May, 2000, in Hillscheid, Germany.

### CLASSIFICATION:

Botanical.—A hybrid of the species Pelargonium peltatum L'Héritier ex Aiton

Commercial.—Ivy-leafed geranium, cv. ‘Fisblizdark’

### INFLORESCENCE:

Umbel:

Shape.—Almost semi-spherically, somewhat irregular.

Average diameter.—119 mm.

Average depth.—50 mm.

Peduncle length.—133 mm.

Peduncle color.—Medium green, RHS 143 A to 143 B.

Pedicel length.—35 mm, with ‘joint/swelling’.

Pedicel color.—Mainly dark-violet, RHS 59A -61 A.

Number of flowers per umbel.—About 7-10.

Corolla:

Average diameter.—49 mm.

Form.—Single-type.

Shape.—Nearly round outline.

Number of petals.—5.

Color (general tonality from a distance of three meters).—Dark red.

Color of upper petals.—RHS 46 A.

Markings of upper petals.—Black stripes (veins) at the base of upper petals.

Color of lower petals.—RHS 46 A.

Color of lower surface of petals.—RHS 46 B to 53 B.

Color of sepals.—Grass-green, RHS 143 A.

Number of sepals.—5.

Bud: (just prior to petals unfolding)

Shape.—Narrow elliptic to asymmetric.

Sepal color (lower part).—Grass-green, RHS 143 A.

Petal color (upper part).—Deep red, RHS 46 A-B.

Length.—19 mm.

Width.—8 mm.

Reproductive organs:

Androecium.—7 fertile anthers, with whitish filaments, orange pollen, the lower side of the mature (open) anther is black.

Gynoecium.—5-6-lobed, purple stigma, RHS 61 B, whitish style.

Fertility/seed set.—Weak seed set through the summer, more seeds are developed from late summer to autumn.

Spring flowering response period: In Hillscheid, Germany, in 2000, plants had on average 2.1 flowers opened 12 weeks after planting of rooted cuttings (pinched plants).

Outdoor flower production: Rich and continuously flowering, the flower count in Hillscheid, Germany, indicated about 4-5 inflorescence per plant in mid May.

Durability: Relatively good shatter resistance for a single-flowered variety, good rain resistance, petal color may slightly fade when maturing.

### PLANT:

Foliage:

Shape.—Ivy-shaped, nearly closed base, distinct lobes, and glossy surface.

Margin.—Entire, apart from the lobes.

Size of leaf.—75 mm.

Color of upper surface.—Medium to dark green, approximately RHS 137 A to 137 B.

Color of lower surface.—Closest to RHS 137 C.

Color of zonation.—Brown, near RHS 166 A, forming a narrow ring.

Tolerance of botrytis.—Good, due to the smooth, glossy surface.

General appearance and form:

Internode length.—45-55 mm.

Branching pattern.—6.6 branches.

Length of branches.—65 cm (15 week old plants in Langley, Canada) 100-110 cm (in mid September in Hillscheid, Germany 31 weeks old).

I claim:

1. A new and distinct cultivar of geranium plant named ‘Fisblizdark’, as described and illustrated.
